(19) United States
(12) Patent Application Publication
(10) Pub. No.: US 2004/0039241 A1
(43) Pub. Date: Feb. 26, 2004

(54) Integrated distribution and communication process and algorithm for providing, handling, distributing or generating reports regarding radioactive pharmaceuticals

(75) Inventors: Bretten H. Whittacre, Las Vegas, NV (US); Troy Curnutt, Las Vegas, NV (US)

Correspondence Address: ROBERT J. SCHAAP., SUITE 188, 21241 VENTURA BOULEVARD, WOODLAND HILLS, CA, 91360-2109, US

(73) Assignee: BIODOSE LLC

(21) Appl. No.: 10/447,726

(22) Filed: May 28, 2003

Related U.S. Application Data
(63) Continuation-in-part of application No. 10/283,480, filed on Oct. 29, 2002.
(60) Provisional application No. 60/384,501, filed on May 29, 2002.

(51) Int. Cl. A61N 5/00

(52) U.S. Cl. 600/1

(57) ABSTRACT

An integrated process and algorithm enabling that process in the handling, providing, distribution or generating reports relating to radioactive isotope irradiated nuclear medicine, and particularly, radioactive pharmaceuticals, including maintenance of data with integrated data communications occurring between at least two or more databases, such as that, for example, a radioactive pharmaceutical supplier and a user of said radioactive pharmaceuticals. Acquisition of data by the pharmacy or the end user can be automatically electronically transmitted to the other, and the required governmental reports dealing with radioactive pharmaceuticals can then be generated automatically acquired data and transmitted to required governmental agencies. The algorithm and process also provide for transmitting data to and receiving process data from associated third parties necessary in connection therewith, including, for example, quality control personnel and the like. The process and algorithm also provide for transmitting data from a remote source and transmitting data to a remote source via non-hard-wired communication.

### BACKGROUND OF THE INVENTION

1. Field of the Invention

This invention relates in general to certain new and useful improvements in an algorithm and process allowing for integrated distribution and communication regarding preparation and delivery or status of materials and transmitting data between databases of two or more organizations dealing with the use and/or administration of radioactive pharmaceuticals. More particularly, the invention involves a process and algorithm allowing for distribution or communication in the providing, handling, distributing and determining status of these radioactive pharmaceuticals between, for example, a pharmacy and a user of the radioactive pharmaceuticals. Further, the method and algorithm allow for the generation of reports on a periodic basis with a relatively minimal amount of additional manual intervention.

2. Brief Description of Related Art

In recent years, the field of nuclear medicine has relied more heavily upon the use of radioactive pharmaceuticals, primarily for diagnostic purposes, but for other purposes, such as treatment purposes, as well. Generally, radioactive pharmaceuticals are introduced into a patient's blood stream, and allowed to be carried to one or more organs of the body which are to be examined. In this way, it is possible to specifically locate tumors or other dysfunction causing conditions.

Also, in the recent past, it has been found that certain tumors, and other dysfunction causing conditions, will not become visually apparent from presently available diagnosing techniques, such as magnetic resonance imaging and computer tomography. However, it has been found that these conditions will become visually apparent when radioactive dies are lodged or introduced into the tumors and other tissue in which observation may be necessary. Due to the greater widespread use of radioactive pharmaceuticals, and the potential for radioactive hazard, both in the handling and in the disposition of waste materials, there has been a need for careful control over the use of such materials. In fact, in the United States, both the federal government and the various state governments have levied numerous regulations controlling the use and disposition of these radioactive materials.

Inasmuch as the pharmacies are essentially the suppliers of the radioactive pharmaceuticals they are inherently in a position to provide data regarding the actual use and, to some extent, the disposition of these radioactive pharmaceuticals. Hence, the various governmental agencies responsible for the control of radioactive pharmaceuticals rely upon this data and are demanding of fairly accurate data regarding these radioactive pharmaceuticals. The handling and disposition of radioactive pharmaceuticals creates numerous problems which are not at all inherent in the use of a standard pharmaceutical. For example, in a radioactive pharmaceutical, concern must be taken to ensure that the radioactivity has not decayed substantially before use thereof by a user. Consequently, there is only a short period of time with some radioactive pharmaceuticals between preparation and actual use by the end user. Although the half life could be increased by using a different radioactive source, a problem arises in the disposition of any remaining portions of that pharmaceutical and also in regard to each of the implements used in contacting that pharmaceutical.

When a standard pharmaceutical product is prepared, the items used in its preparation such as beakers, flasks and the like can be merely washed out or discarded, if desired. In the case of a radioactive pharmaceutical, since these items were in contact with a radioactive source, they too have become radioactive and hence control must be carefully exercised over the disposal thereof. In addition, control must be maintained over the areas in which the radioactive pharmaceuticals were used or were prepared. Consequently, the problems arising out of the use of radioactive pharmaceuticals are far more significant than with other types of products.

It can be understood that there are numerous details involved in the handling and the use of radioactive materials and for that matter in the handling of these materials, or items in contact therewith. As a result, the reporting requirements can be and frequently are extensive and involve the preparation of time consuming and hence expensive reports. Not only have the users and pharmacies become involved in these myriads of reporting requirements, but the pharmacies themselves have had to report ever increasing amounts of information to the various governmental agencies. Hence, many pharmacies have had to hire full time personnel to do nothing more than generate reports.

The pharmaceutical houses which dispense these radioactive materials, are required to account for complete use of the radioactive materials, including the handling of the waste resulting therefrom. These pharmaceutical houses are also required to generate reports to those government agencies which are involved in the regulation thereof. The same holds true, to some extent, for the end users of these radioactive pharmaceuticals, as for example, the hospitals and the physicians and medical centers which are involved in the administration of these radioactive pharmaceuticals.

The pharmacies are typically involved in the business of acquiring or producing a pharmaceutical product and then imparting the desired amount of radiation to that product. They do not provide medical care or administer radioactive pharmaceuticals and do not keep data regarding the administration. However, the pharmacies are required to generate governmental reports which becomes a very time consuming and, therefore, an expensive task.

This increasing widespread use of radioactive pharmaceutical materials has given rise to a number of radioactive pharmaceutical supply facilities which supply the end users, e.g., medical institutions, hospitals and physicians with these radioactive materials. These organizations which primarily deal only in the generation and distribution of radioactive pharmaceuticals, are referred to as “pharmacies.” Therefore, as used here, the term “pharmacy” shall mean and refer to only those organizations who deal in the generation and distribution of radioactive pharmaceuticals and who are, or may be, subject to governmental control in the production and/or distribution of such radioactive pharmaceuticals.

Initially, some of the pharmacies, and for that matter, even the end users, such as hospitals, etc., were originally quite lax in control of the radioactive materials, and in the maintenance of data for generation of reports. Consequently, the U.S. government and the various state governments, have enacted, and even tightened, regulations which require very accurate reporting on a periodic basis.

There have been a few proposed attempts to provide an algorithm and a software program which enables users such as hospitals, clinics, physician's offices and the like to potentially record information relating to the use and disposition of the radioactive pharmaceuticals. These software programs attempted to provide some means for recording information so that the required governmental reports and other reports could be generated. However, these programs were quite lacking as hereinafter described.

There have also been attempts to provide a pharmacy based algorithm and software program which allows pharmacies or other suppliers dealing with radioactive pharmaceuticals to keep track of these radioactive pharmaceutical materials. These pharmacy based algorithms and software programs were similarly lacking, again, for reasons as hereinafter described. However, heretofore, there has been no attempt whatsoever to integrate the pharmacy based algorithm and software program and the end user algorithm and software program so that there was a linking between the two. In addition, there has not been any proposed algorithm or software program which permits a two-way communication between a pharmacy dealing with radioactive pharmaceuticals and a user of those radioactive pharmaceuticals.

With regard to the previous attempts to use data processing techniques for recording information regarding radioactive pharmaceuticals, the prior attempts almost always relied upon a pure storage of information without much attention being given to segregation of data for purposes of auditing and even for that matter, report preparation. Again, these particular programs were designed, primarily, to meet the needs of a particular organization and were not universally adaptable for use by a variety of organizations. Notwithstanding, and although software programs for both pharmacies and users had been available, even though rudimentary, there was still no attempt to ever allow for integration of information between the two.

One of the primary problems with the prior art software programs which have been generated for handling and delivery of radioactive materials by a pharmacy or by a user, is the fact that they were not particularly user-friendly. The operator of the system had to be fairly well experienced in dealing with computers in general, and in switching back and forth between subroutines in complex algorithms. As a simple example, if there were a menu page presented on the screen of a monitor, the operator would have to track the particular page involved, in order to examine details of a routing on that menu page. In many cases, the operator even had to go to additional menu screens in order to find the routine which was needed. Moreover, when the operator finished with one routine, the algorithm did not allow the operator to immediately return to the main screen, with a mere click of a pushbutton switch.

Another one of the problems inherent in the prior art systems is that they were not readily adaptable to changing requirements. Thus, if a governmental agency required a new type of report or an altered report to be generated, this almost necessitated the need for a skilled programmer to input that instruction base necessary for an operator to use. Consequently, the prior art programs were severely lacking in may respects.

There has been a need for a method and algorithm for handling radioactive materials which is designed for enabling communication between the databases regarding these radioactive materials. There has also been a need for a method and algorithm which allows for the maintenance of records, the acquisition of data regarding the use of the radioactive materials and the handling and distribution of these radioactive pharmaceuticals.

There has further been a need both by pharmacies and users of radioactive pharmaceuticals for systems which will allow for the automatic retention of data, segregation of data according to specific functions and materials, and which will also generate reports based on the collected data, all on an automated basis. There has also been a need for systems of this type which could be universally applicable to the collection and segregation of data and generation of reports, based on the activities employed and the functions which are necessary by a pharmacy and an end user of such systems.

There has also been a need by third parties who are authorized to have access and who, from time to time, need access to a database dealing with these radioactive pharmaceuticals. As a simple example, health physicists may have information to introduce into the database or need to extract certain information from that database. Moreover, the health physicist and for that matter, others, may be at a remote site with respect to the one or more databases involved. Heretofore, there was no means to enable someone at a remote site to introduce data into or to extract data from one of the databases.

The present invention provides a unique system which allows communication between a database containing information on radioactive pharmaceuticals and another database similarly containing information on radioactive pharmaceuticals as for example, a database at a pharmacy and a database of the user. The present invention allows for a vertical communication between these two. The present invention further allows for communication with one or more databases from a remote site. In addition, the present invention provides for security in that only those parties having authorization to the databases can access same.

### OBJECTS OF THE INVENTION

It is, therefore, one of the primary objects of the present invention to provide an algorithm and method for allowing integrated distribution and communication for providing, handling and distribution or generation of reports regarding radioactive pharmaceuticals and which enables communication between two or more databases with respect to those radioactive pharmaceuticals.

It is another object of the present invention to provide for a method and algorithm of the type stated which allow acquisition of data from one or more databases and storage of same in the acquiring database with regard to radioactive pharmaceuticals and which can be automatically electronically transmitted between one another.

It is a further object of the present invention to provide an algorithm of the type stated which allows for highly efficient and generally error free communication between a supplier of radioactive pharmaceuticals and a user of such radioactive pharmaceuticals.

It is an additional object of the present invention to provide for access to one or more databases which contain information regarding radioactive pharmaceuticals from a remote site through a database at that remote site as for example, a database contained within a handheld transmitting and receiving unit.

It is also an object of the present invention to provide an algorithm and a software program generated thereby and a method enabled by the algorithm and software program for handling and maintaining records with regard to radioactive pharmaceuticals and which is universally adapted to a variety of situations and a variety of activities and the handling, preparation and dealing in general with radioactive pharmaceuticals.

It is still another object of the present invention to provide a method for the gathering of data, storing of data and transmission of data involving radioactive pharmaceuticals which allows for segregation of that data according to some pre-established pattern such that the data can be readily and easily accessed for use by one or more parties and for the generation of reports based on the use of such data.

It is still a further object of the present invention to provide an algorithm and method of the type stated for handling and maintaining records of radioactive pharmaceutical products on a highly efficient automated basis which previously required manual intervention for transferring data from one database to another regarding such radioactive pharmaceuticals.

It is a further salient object of the present invention to provide an algorithm and program of the type stated, which can be universally adapted to a variety of situations and a variety of activities in the handling, preparation and delivery of radioactive materials which may be used.

It is another salient object of the present invention to provide an algorithm and a program of the type stated, for a pharmacy providing radioactive pharmaceuticals, which can be fully automated and where reports can be generated with very little manual attention on a periodic basis, and containing that information precisely categorized and specified as required by various governmental agencies.

With the above and other objects in view, my invention resides in the novel features of form, construction, arrangement and combination of parts and components presently described and pointed out in the claims.

### BRIEF SUMMARY OF THE INVENTION

The present invention, in a broad sense, relates to an algorithm for establishing an electronic, bi-directional communication between at least a first database and a second database for the producing, delivery and monitoring of an isotope irradiated nuclear medicine. More specifically, the isotope irradiated nuclear medicine is used in a broad sense to refer to both the radioactive pharmaceutical which is administered to a patient and to the implements used in the production, delivery and administration of that radioactive isotope. The first database may be any database as for example, a fixed database at a fixed location, e.g., a clinic or physician's office, and the second database may be a database, also at any location, as for example, a pharmacy. In either case, the databases can be remote databases, as for example, a database in a handheld calculator device with storage capability.

The algorithm of the invention provides for the sending of information regarding the isotope irradiated nuclear medicine from the first database to the second database. This information may be data pertaining to the production or delivery of the isotope irradiated nuclear medicine or the use thereof or in the handling and disposition of any implement used in connection therewith and which implement might have been also irradiated as a result thereof. The process thereafter calls for the termination of the sending of information of this database. As indicated previously, the handling of isotope irradiated nuclear medicines is entirely different from the handling of other pharmaceuticals and for that matter, other materials, whether or not used in the health care field. There has been no effective algorithm heretofore which allows for sending of information between two individual databases. These databases, in the example given above, were a pharmacy and a user, such as a hospital, clinic or the like. However, and notwithstanding, there has not been any effective communication between two or more databases of independent organizations in the past.

The communication link which is established is bi-directional, as aforesaid. Because of the sensitivity of the subject matter which can be transmitted or received, usually one or both databases will contain in the algorithm a check on authorization in order to determine whether or not information requested at one database can be transmitted from the other. If such information is not authorized then communication would be immediately stopped.

The algorithm of the invention also allows for the acknowledgment of receipt of information. Thus, for example, if a user inquires of a pharmacy as to the status of an order, the database at the pharmacy can automatically send back an acknowledgment that the request has been received and at a later point the request can be answered.

In another aspect of the invention, if an inquiry is made of a second database from a first database, the personnel at the second database can automatically preclude any response to an inquiry until the second database makes an inquiry of the first database. When the inquiry made of the first database is received at the second database, then the second database can provide an answer to the original inquiry made of that second database.

The algorithm of the invention includes several major steps for operation of a software program using the principle of this algorithm. Thus, the algorithm may establish a step for establishing a link between the two databases. This link would be based on the type of communication which is received obviously, a wireless communication would require a different link than a hardwire communication. A communication from a computer would require a different link than a communication with a person using voice transmission.

The algorithm would next provide a step for transmitting information about the isotope irradiated nuclear medicine related item from one database to the other. The algorithm would provide for electronically determining the mode of information handling at the second database and would also provide for transmitting information from the first database to the second database. Finally, the algorithm can provide for automatic termination of messaging.

The algorithm of the invention is only briefly described at this point but may well include other numerous steps. For example, one step which can be included is that to determine if the information being transmitted to the second database is already in the second database and disregarding that information if the same is in the second database.

Also in a broad aspect of the invention, which is primarily useful where one of the databases is that of a user, such as a hospital or clinic, etc., and the other is a pharmacy or similar radioactive pharmaceutical supplier, there are certain common elements which are mandated by various governmental agencies. One of those elements which is mandated is that of quality control. In effect, anyone dealing with the radioactive isotope and irradiated material must maintain records of contact and the disposition of the radioactive pharmaceutical, as well as any implement used in connection therewith.

The term “implement”, as used in connection with the radioactive pharmaceutical, is that which refers to a container for the pharmaceutical, a means for injecting the pharmaceutical, a means for disposing of the pharmaceutical and the like. The term implement also refers to surface areas on which the radioactive pharmaceutical may have been placed or stored. Again, data is required to be maintained on all of these areas and for that matter the personnel who deal with these radioactive pharmaceuticals. The term “quality control” is therefore used in this broad sense to refer to all of the activities of monitoring and maintaining data with regard to the radioactive pharmaceuticals and the implements used therewith.

As indicated above, in the algorithm there is a requirement for quality control. While quality control may be initiated at the beginning of any procedure, various quality controls are required throughout the procedure, largely due to the fact that radioactive pharmaceuticals are involved.

After initial quality control, the pharmacy or other supplier will then determine whether the requested pharmaceutical is in a bulk storage then the proper allocated amount is withdrawn and introduced into another vessel for delivery and ultimate administration. If the required amount of radioactive pharmaceutical does not exist, then it may have to be eluted. The term “eluted” or “elution” as used herein refers to the process of both mixing the necessary ingredients to produce the pharmaceutical and thereafter to irradiate that pharmaceutical with a suitable radioactive isotope so that there is a desired amount of radioactivity in the radioactive pharmaceutical. In the latter step, care must be taken to account for delivery time and time of administration because of potential rapid decay rates.

After production of the radioactive pharmaceutical, the pharmaceutical itself must be packaged properly for delivery, as aforesaid. Thereafter, maintenance of records for administration of the pharmaceutical to a patient must thereafter be maintained.

As indicated previously, one of the important facets of the algorithm of the present invention is the maintenance of data for producing the required reports to governmental agencies and others. In this case, for example, the data must be segregated appropriately and placed into individual data banks so that all of the necessary data can be rapidly and easily addressed for generation of reports. Moreover, the algorithm of the invention also provides for the actual generation of the reports thereby saving an otherwise substantial amount of manual attention required for preparing these reports.

One of the important aspects of the present invention is the fact that all of the data which is necessary to be maintained could be transmitted bi-directionally between the individual databases. Not only has there not been a convenient and computer friendly algorithm and program for handling and disposition of radioactive pharmaceuticals, there has been no attempt whatsoever to provide for the bi-directional vertical or for that matter even horizontal communication between data banks dealing with radioactive pharmaceuticals.

One of the important aspects of the invention is the fact that there is a complete record keeping in all facets of the radioactive pharmaceutical such that one having authorization can inquire as to the status of, for example, an order or the like.

At the start of any communication process, it is, of course, necessary to ensure that there is a basis of communication between the databases, again taking the pharmacy and the end user as an example of two databases, so that data can be transmitted between the pharmacy and user. For this purpose, email is one of the effective electronic communication links, and thus, email addresses are set up appropriately for each of the parties. During an average day of operation, the pharmacy will check its email or other mail system for information, in order to determine if there have been any orders or similar communications from the end users, health physicists or others. Other means of potential communication must also be examined, for example, bidirectional communication, using mail, fax letters, and the like.

If the communication indeed is an electronic communication, two individual and independent paths of activities are created. In the first case, an order is sent to a defined address, as for example, on an email. An email server associated therewith will parse the information and insert that information into the pharmacy database, when that information is sent from a customer or end user. At that point, the pharmacy will assign a confirmation number, and send a confirmation of receipt of information back to the sender, such as the customer. Someone at the pharmacy must examine and determine if there is new information on the server. If there is no new information, then the procedure is stopped. If there is information, one must parse that information and integrate the same into a relevant database.

Simultaneously with the first step of enabling communication between the pharmacy and the customer, a separate track is followed for generating information to be ultimately delivered to a health physicist. In this case, the information which is sent to the pharmacy by the customer is also sent to the health physicist. The health physicist also has a server which will parse information. Moreover, the health physicist communication system will notify a particular party at the health physicist, of information which may be present. The health physicist will then send a confirmation of receipt of information to the customer's email address or to the pharmacy's email address, or both.

The health physicist must also look for new information on its server. If there is no new information, then the process will stop. If there is new information, the system of the health professional will parse that information and integrate the same into the health physicist's database. At that point, the system of the health physicist will notify the customer or the pharmacy, or both, of the receipt of the new data.

The integrated system is a bidirectional system between, e.g., the pharmacy and the end user, such as the physician or hospital, where the latter will send information to the email server of the pharmacy at a specific address and vice versa. That email server will parse information to the right location, as for example, a database table. In this case, a table may have already been established in the pharmacy's system and the return data will be segregated into the proper locations in that table.

This present invention thereby provides a unique and novel integrated distribution system, process and algorithm for providing and distributing radioactive pharmaceuticals and generating of reports therefor, which thereby fulfills all of the above-identified objects. Other objects which will become more fully apparent from the consideration of the forms in which the invention may be embodied. One of these forms is more fully illustrated in the accompanying drawings and described in the following detailed description of the invention. However, it should be understood that the accompanying drawings and this detailed description are set forth only for purposes of illustrating the general principles of the invention.

This invention possesses many other advantages and has other purposes which may be made more clearly apparent from a consideration of the forms in which it may be embodied. These forms are shown in the drawings forming a part of and accompanying the present specification. They will now be described in detail for purposes of illustrating the general principles of the invention. However, it is to be understood that the following detailed description and the accompanying drawings are not to be taken in a limiting sense.

### BRIEF DESCRIPTION OF THE DRAWINGS

Having thus described the invention in general terms, reference will now be made to the accompanying drawings (9 sheets) in which:

FIG. 1 is a schematic block diagram showing transmission of data between a first database and a second database and a database at a remote source;

FIG. 2 is a schematic block diagram showing the arrangement for integrated communication between three individual fixed databases and a database at a remote source;

FIG. 3 is a generalized schematic block diagram showing the process involved in using the algorithm of the present invention in a communication between a pharmacy and a user;

FIGS. 4A and 4B are a composite flow diagram showing an example of an algorithm allowing for the establishment of a communication link between a sender and a receiver in accordance with the present invention;

FIGS. 5A and 5B are a composite flow diagram showing an example of an algorithm in which ordering occurs between a patient facility and a supplier;

FIG. 6 is a schematic flow diagram showing an example of an algorithm for ordering by a patient facility to a supplier; and

FIGS. 7A and 7B are a composite flow diagram showing an example of an algorithm for enabling ordering by a patient facility and with inquiry by a supplier before the filling of the order.

### DETAILED DESCRIPTION OF A PREFERRED EMBODIMENT

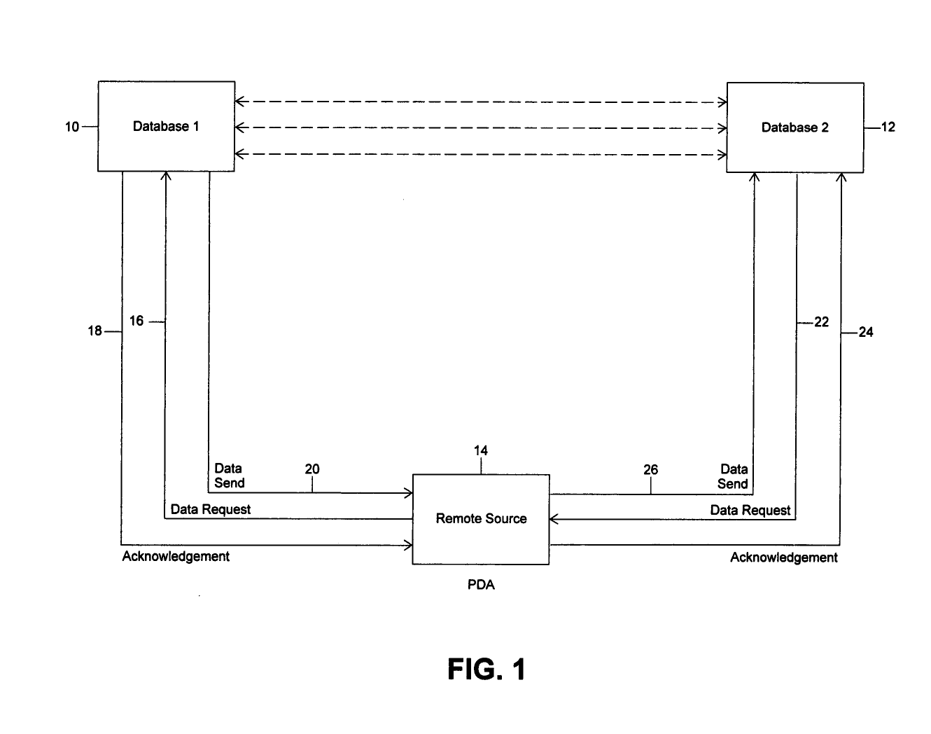

Referring now in more detail and by reference characters to the drawings, FIG. 1 illustrates a schematic block diagram showing the integrated communication links between a first database and a second database as well as a remote source allowing for the distribution, handling and administration of radioactive isotope irradiated pharmaceuticals, such as radioactive pharmaceuticals.

In the arrangement of FIG. 1, there is provided a first database 10 communicating with a second database 12 and both of which provide for communication between a remote source 14. In the arrangement as illustrated, it can be seen that the remote source 14, for example, may send a request for information over a communication path 16 to the first database 10 and the first database 10 may then send an acknowledgment over a communication path 18. Thereafter, the first database 10 may send that data over a communication path 20. In like manner, the second database 12 can send a request for information over a communication path 22 with an acknowledgment from the remote source 14 to the second database 12 over a communication path 24. Finally, the data can be sent over a communication path 26 to the second database 12.

FIG. 1 illustrates three dotted lines between the first database and the second database. In effect, these lines represent the communication paths, such as the communications 16, 18 and 20 which occur between the first database and the remote source, 14, for example. It should be understood that these various communications would all occur over a single link in temporal relationship. It should also be recognized that the arrows in FIG. 1 could be reversed so that, for example, instead of the second database 20 sending a request 22, to the remote source 14, that remote source 14 could send the request 22 to the second database 12 with an acknowledgment from the second database and the transmission of data from the second database 12 to the remote source 14.

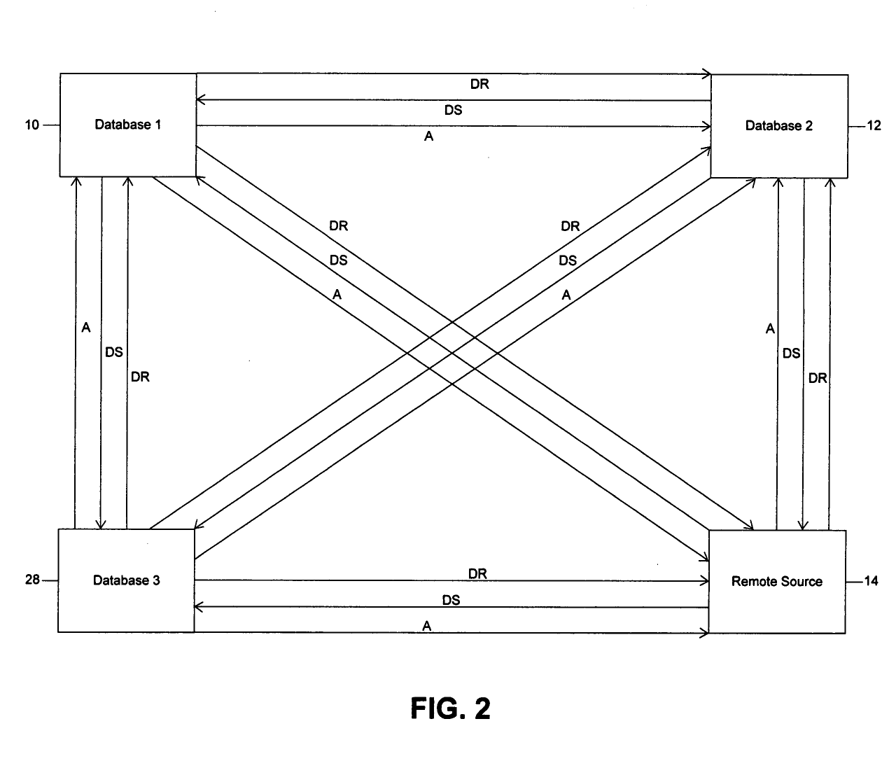

FIG. 2 illustrates an arrangement in which there are three databases, such as the database 10, the database 12 and the remote source 14 with an additional database 28. It can-be observed, by reference to FIG. 2, that the same three communications which occur between the various databases in FIG. 1 are also established in FIG. 2. Moreover, there are communications between each of the databases including the database of the remote source 14. Thus, there are three additional sets of communication lines between the various databases and the remote source in FIG. 2. Again, the direction of the arrows in FIG. 2 can be reversed so that communication takes place in the opposite direction, as well.

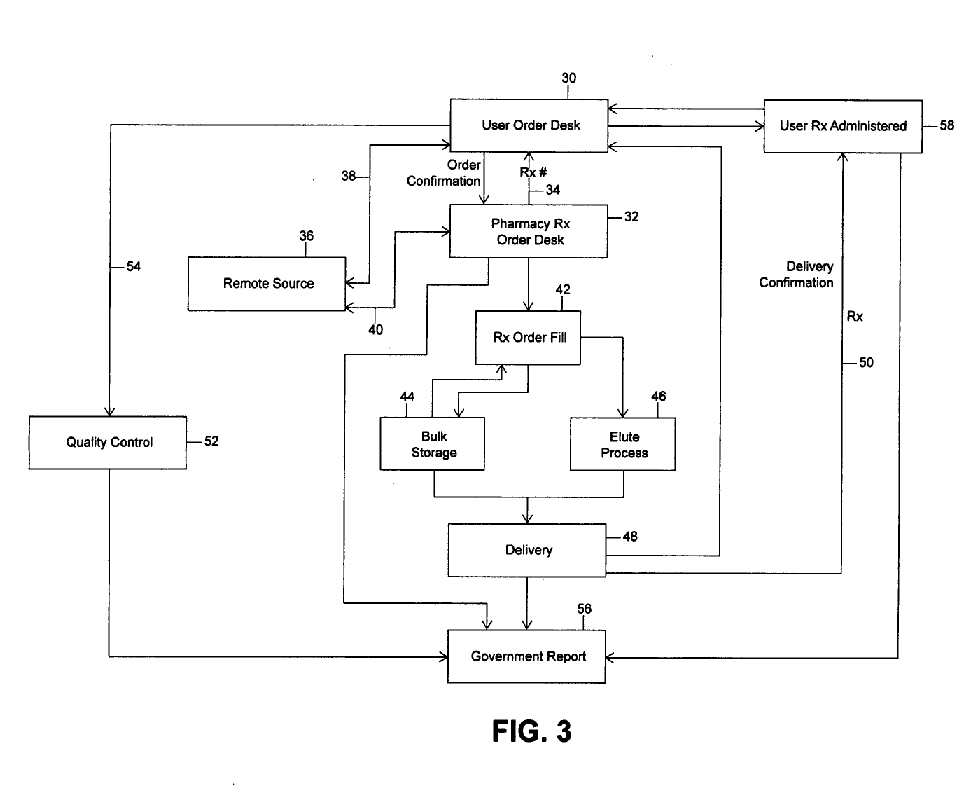

FIG. 3 illustrates a broad schematic diagram showing the integration of a database for a user and a database for a pharmacy with regard to the ordering and disposition of a radioactive pharmaceutical. In this case, the user has an order desk 30 and can communicate with a radioactive pharmaceutical supplier, such as a pharmacy, having an order desk 32, over a pair of communication lines 34. In like manner, both the user and the pharmacy can have a bi-directional electronic communication with a remote source 36 via links 38 and 40. The remote source may adopt the form of, for example, the database of a health physicist.

When an order is submitted to the pharmacy 32 over the links 34, that pharmacy will attempt to fill the order at step 42. The order will be filled by first determining whether or not the required pharmaceutical is in bulk storage at step 44. If it is not in bulk storage, the process will advise the party filling the order that they must use the elute process 46. The elution step was described above and is hereinafter described in more detail herein. In any event, after the order is filled, it is sent to a delivery station 48 where it can then be delivered to the user through a delivery 50. In this case, the delivery is a physical delivery, whereas the other links were either wireless or hardwired communication links.

One of the important aspects of the algorithm of the present invention is that of maintaining data for submission of government reports. That data and particularly data relating to quality control is effectuated at step 52. It can be observed that data from the user is submitted to the quality control step at link 54 and data from the quality control desk is then sent to a database for government reports at 56. In like manner, the government report database receives information from the pharmacy desk 32 and the delivery step 48, as well as reply information from the user at step 58.

The block diagram of FIG. 3 more fully sets forth those steps which are frequently employed with the algorithm of the invention in the ordering of a radioactive pharmaceutical. Inasmuch as each pharmaceutical must be accompanied by data for a governmental report, the algorithm of the invention provides for automatic generation of that data and retention of the data into an appropriate database for the preparation of that governmental report. Frequently, the database containing this information may exist with the user of the radioactive pharmaceutical, such as the hospital or clinic. However, certain other information could be retained in the database of the pharmacy. Moreover, information of the health physicist is usually required for the generation of the governmental report since quality control, as described above, is an important factor for inclusion in each of these governmental reports.

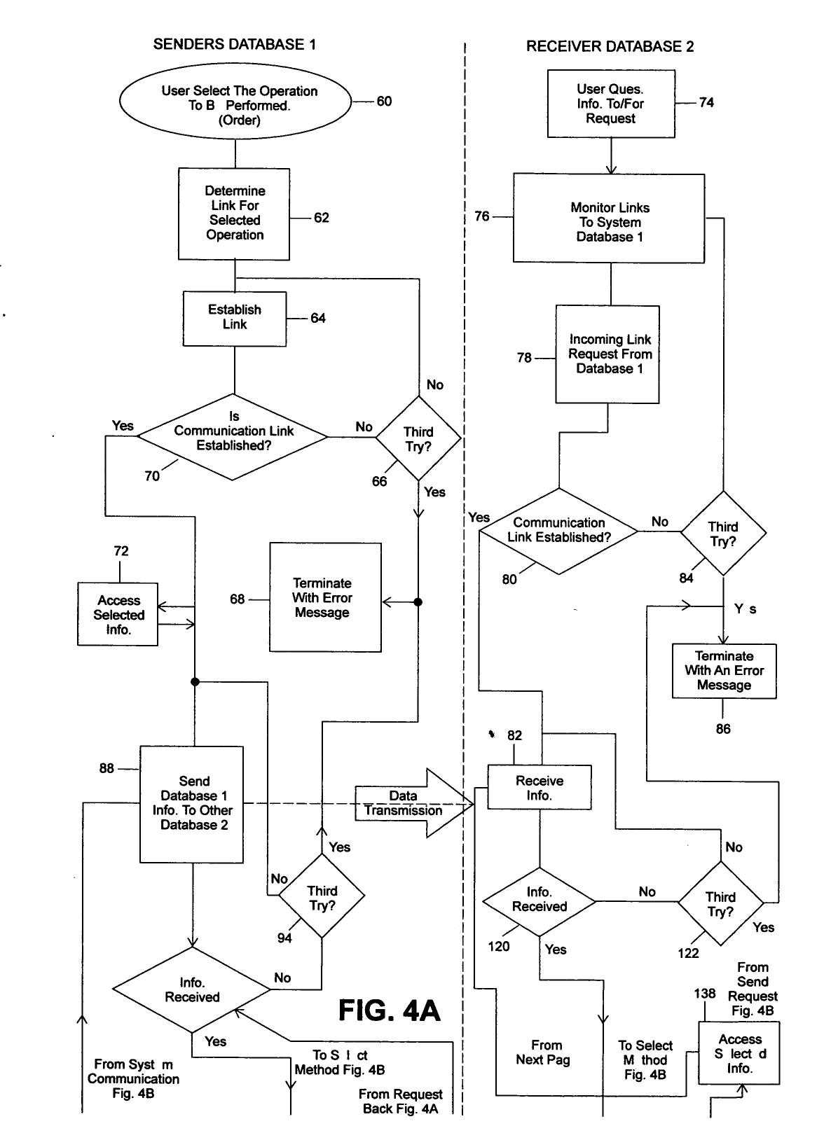

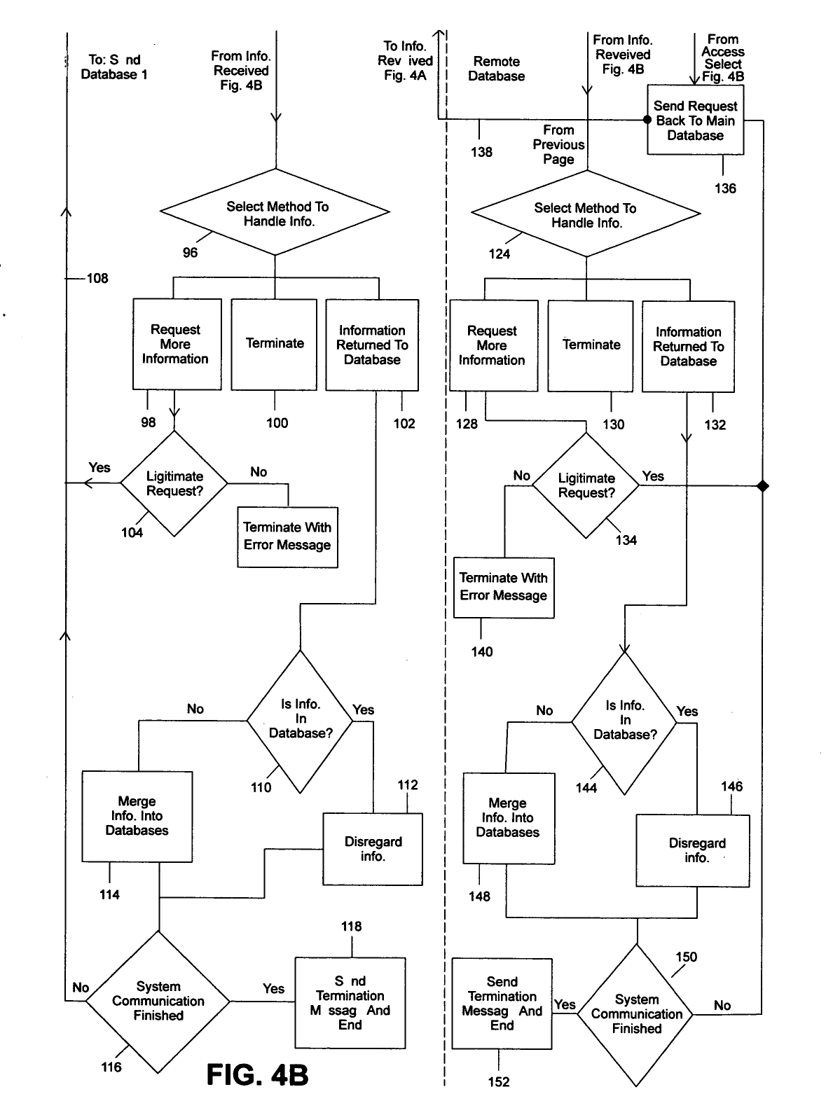

FIGS. 4A and 4B illustrate a somewhat generic flow diagram showing the steps of an algorithm which could be used for the generation of a computer program and hence a method in accordance with the present invention. In this case, the algorithm illustrates the flow of data and the communication links between a sender and a receiver. The sender is identified as having database 1 and the receiver as having database-2. Obviously, the algorithm is bi-directional such that the receiver could become a sender and the sender could become a receiver. This algorithm only shows a two-way communication although it would be obvious that it could be adapted for other communication as well. In essence, this same algorithm would be used if the sender were attempting to communicate with a third database, as for example, the database 3 at 28 or for that matter a remote source such as the remote source 14 in FIGS. 1 and 2.

In the sender algorithm, the user will determine and select the operation to be performed at step 60. In this case, for purposes of illustrating the example, the user is shown as sending an order to the receiver. The algorithm thereafter determines the link which is to be used for a selected operation at step 62. For example, data from a remote source, as for example, a handheld computing device, may be a wireless communication and the algorithm would have to establish a wireless communication path. Again, a different communication path would be established for verbal communications and still a different path for information sent by e-mail.

The link which is determined at step 62 is thereafter established at step 64. The algorithm provides for a determination of whether or not there have been multiple attempts to establish a link at step 66. In this case, and in the algorithm of the invention, the number of attempts is three. Thus, if this was the third try or attempt to establish a link, the program would continue at 68. If this was not the third attempt, the algorithm would provide for termination of the attempt with an error message indicated to the party sending the message. If this was less than the third attempt to establish a link, which is determined at step 70, the algorithm will then proceed to accessing the selected information at step 72.

Simultaneously with the steps occurring with the sender, there are several steps associated with the algorithm which are occurring with respect to the receiver of such communication. In this case, the user, such as the user at step 60, queues information to or from the second database, in the form of a request, at step 74. Again this database would monitor the connected link to the system and particularly database-1 at step 76. The algorithm at the receivers database-2 would receive an incoming link request from the database-1 at step 78.

At this point, an inquiry is made at step 80 as to whether or not the communication link between the two databases has been established. If the communication link has been established at step 80 then the algorithm provides for the receipt of information at step 82 from the first database. The algorithm will also determine whether or not this has been the third attempt to establish a link at step 84. If this is the third attempt, then the algorithm will cause the method to terminate with an error message at step 86. Otherwise, the algorithm will allow for the receipt of information at step 82.

When the two sets of algorithms reach this point, there will be established a path for communication from the first database to the second database. When information is accessed at step 72 in the database-1, that information is sent to database-1 for submission to database-2 at step 88. At this point, an information path or data transmission path is established between step 88 and step 82 as shown in FIG. 4A of the drawings. Thus, when database-1 desires to receive information from database-1, that information is transmitted to database-2 over the communication path 90, as shown in FIG. 4A. It is to be noted, that with respect to the various steps of the algorithm, and the flow of activities, that all of the various elements are connected by a solid line. The data path is represented by a dotted line.

Returning again to the sending database-1, it can be observed that the algorithm provides a step 92 to determine if information was received from the second database. Again, at step 94 a determination is made as to whether this is the third attempt for receipt of information and if so, then the algorithm will provide for termination with and error message at 68. If it has not been the third attempt, then the algorithm will return back to step 88 so information can again be sent from the first database to the second database.

The algorithm continues so that if information has been received at step 92, it will continue to select a desired method to handle the transfer of information at step 96 and as best shown in FIG. 4B. In effect, there are three possibilities for selecting a method to handle the information which is received. The first of these methods relies on a request for more information at step 98, or to terminate at step 100, or otherwise to return information to the databases, at step 102. Again if the method is to request more information, then a determination is made at step 104 as to whether or not this is a legitimate request. If it is not a legitimate request, the algorithm will call for termination with an error message at step 106 and if it is a legitimate request, then another request will be sent over path 108 from database-1 to database-2 at step 88 in FIG. 4A.

One of the important aspects of the present invention is the fact that the information is sensitive since it contains much personal information both of patients and of the user and, for that matter, of the pharmacy. Consequently, determinations as to whether a request is legitimate can be made throughout the algorithm of the invention.

If information is to be returned to one of the databases for insertion into those databases at step 102, another determination must be made at step 110 as to whether that information is already in the database. If that information is present in the database, then the algorithm calls for a disregarding of that information at step 112. However, if the information is not in the database then the algorithm permits insertion of that information at step 114. At this point, a decision is made as to whether or not communication has been finished. If it has, then there will be a termination at step 118 and if communication is not finished, then the algorithm will return to step 88 over the link 108.

Returning again to the receiver database-2, if information has been received at step 82, a determination will be made at step 120 as to whether or not that information has been received. In particular, the algorithm will determine whether information from database-1 has been received at step 82. If it has been determined at step 120 that the information has not been received, a determination will be made at step 122 as to whether this is the third attempt. If so, then the algorithm will root back to step 86 and terminate with an error message.

If information has been received, then the algorithm will proceed to step 124 providing for the selecting of the method to handle the information. Again, a decision is made at step 124, similar to a decision at step 96, to select the proper method to handle information. Again, the algorithm may request more information at step 128 or it may terminate the transmission at step 130 or it may provide for return of information to the databases at step 132. At this point, the algorithm, as shown in FIG. 4B, generally parallels the portion of the algorithm for handling information at database-1.

The algorithm again determines whether or not the request for information from database-2 is a legitimate request at step 134. In this respect, it can be observed that the algorithm actually operates in both ways such that the first database can request information from the second database and the second database cause a transfer of information to the first database, or otherwise, that the second database may request information from the first database and the first database causes a transfer of information to the second database. Moreover, the algorithm can operate such that the first database may request information and the second database operates in a manner to ask for information from the first database before the second database sends the requested information to the first database. When the first database has sent information back to the second database answering the inquiry of the second database, then the second database can allow for transfer of the information originally sought at the first database.

Frequently, human personnel may be involved in the operation of the algorithm at that point to determine if the information is sufficient. In many cases, the information being transferred is electronic such that human intervention is not required. As a simple example, if the second database is attempting to acquire information with regard to administration of a pharmaceutical, an inquiry may be made at the first database. If the inquiry is not specific as to which patient, the first database may attempt to inform the second database that more information is needed as to the specific patient. When that information is returned from the second database, then the first database can provide the information originally sought.

If the algorithm determines at step 134 that there is a legitimate request for information at the first database, then the algorithm will proceed to send a request back to the main database at step 136. That request will move to step 138 in FIG. 4A for accessing the selected information. The algorithm then allows for return to step 82 for receipt of that information from step 136. It can also be observed that if information is sent over the data path 90 from step 88 to step 82, the second algorithm will provide for an acknowledgment of receipt of that information. That receipt notification is sent from the second database 136 over data path 138 with that acknowledgment being received at step 92 in FIG. 4A. If the request at step 134 was not a legitimate request the algorithm will call for termination with an error message at step 140.

If the information returned for introduction into the databases at step 132 is selected, the algorithm will further determine whether that information is already in the databases at step 144. If the information is in the database it will regard that information at step 146. If not, then the information will be merged into the databases at step 148. At this point, a determination is made at step 150 as to whether or not communication is finished. If communication has been finished, a termination message will be provided at step 152 and the information transfer will end. If there has not been a finishing of the communication, then the algorithm will return back to step 136 to send a request back to the main database, namely database-1 for more information.

In accordance with the generic version, as shown in FIGS. 4A and 4B, it can be observed that there can be a one-way communication as, for example, from database-1 to database-2 or from database-2 to database-1. The same algorithm would exist for other databases, such as a database-3, as shown in FIG. 2. Moreover, the same algorithm would be operable with respect to portable databases, such as one in a handheld device. The algorithm also allows for two-way communication, such that the first database can make an inquiry and the second database can similarly make an inquiry before answering the inquiry of the first database. The algorithm is also effective with differing types of communication links.

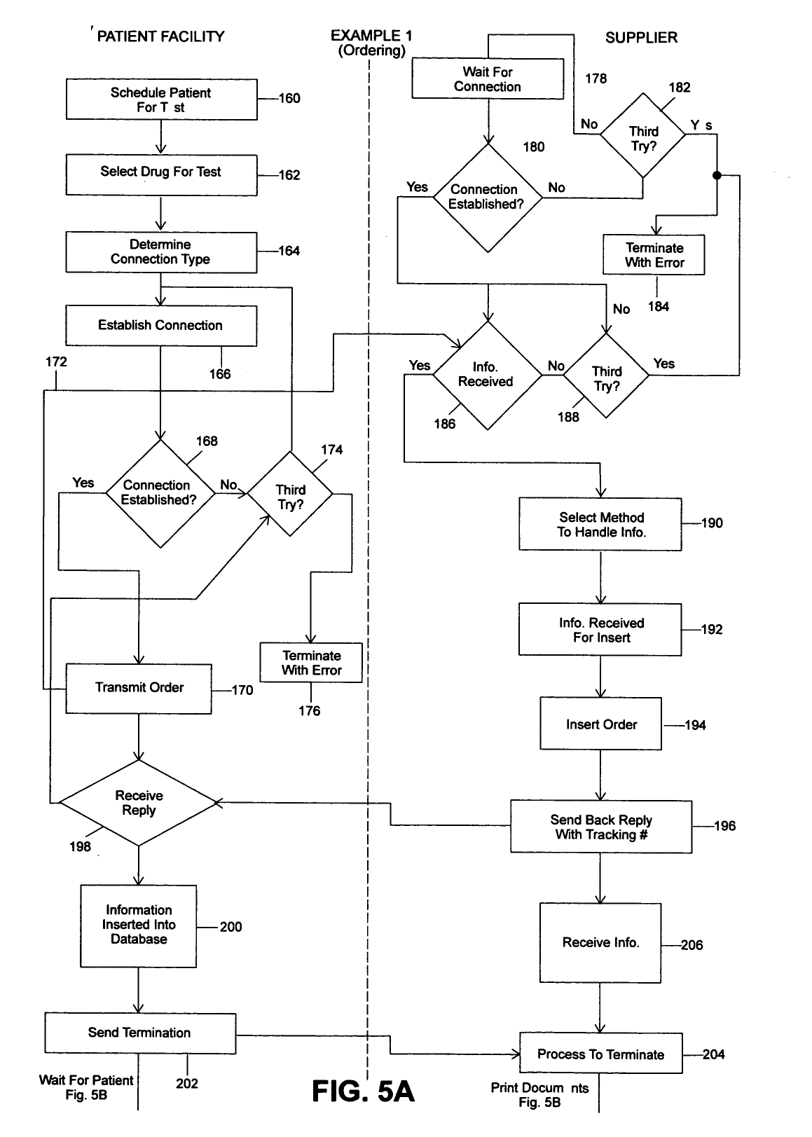

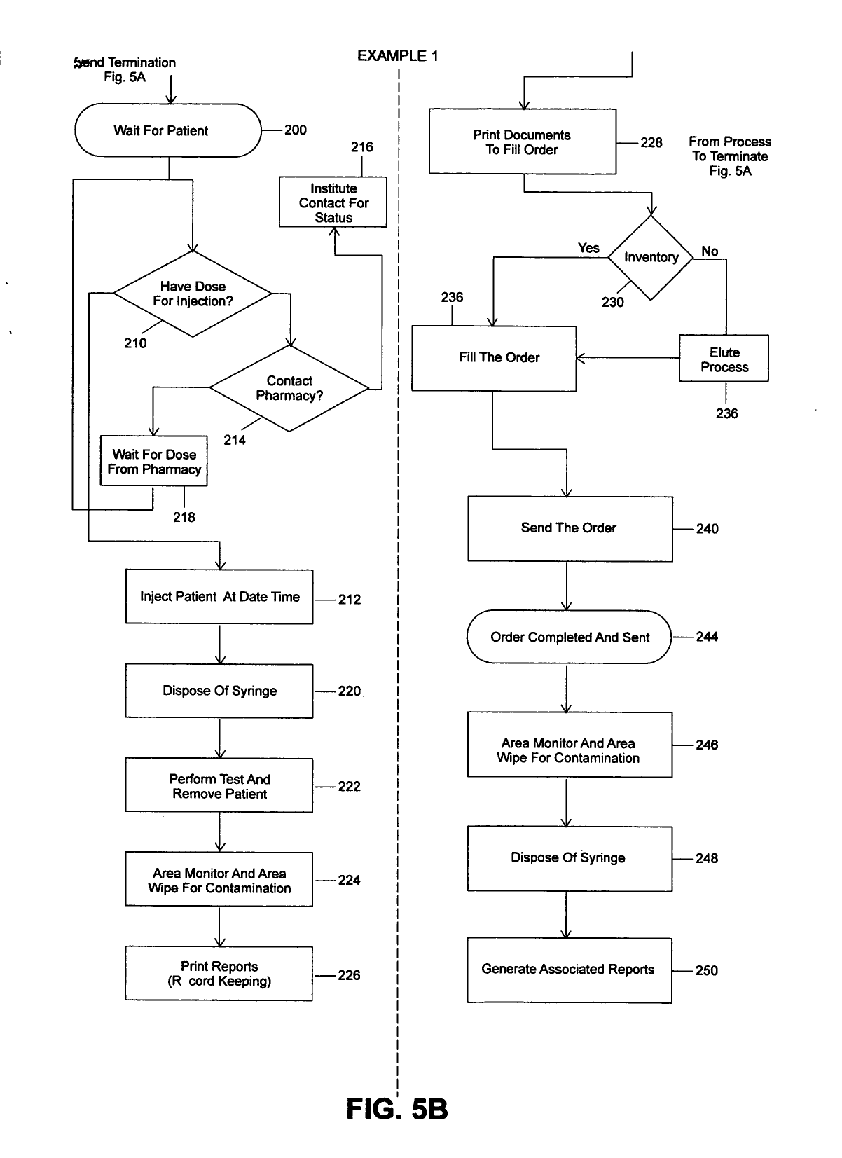

FIG. 5 comprised of FIGS. 5A and 5B represents an example of ordering an isotope irradiated nuclear medicine. In this case, the algorithm starts with the scheduling of a patient for a test or for the administration of a therapeutic radioactive pharmaceutical, at step 160. At that point, a drug or pharmaceutical must be selected for that test or therapy at step 162. The algorithm also provides for determining the type of connection between the patient facility on the left hand side and the supplier facility on the right hand side of FIGS. 5A and 5B. This connection type is determined at step 164. Thereafter, the algorithm provides for establishing the connection at step 166.

The algorithm then continues and determines whether a connection has been established at step 168. If a connection has been established, it will cause the transmitting of an order at step 170. That order will be transmitted to the supplier over a link 172, in a manner as hereinafter described. The algorithm will also provide for a determination as to whether or not the establishment of the connection is based on a number of attempts and for example the third attempt, at step 174. If there have been three attempts, then the algorithm will cause a termination of communication with an error message at step 176.

The algorithm on the supplier side, such as the pharmacy, will cause the database of that supplier to wait for a connection at step 178. A determination is then made at step 180 as to whether or not a connection is established on the supplier side. Again, it is necessary to determine whether or not this is the third attempt at a connection at step 182. If it is the third attempt, then the algorithm will automatically terminate with an error message at step 184. If it is not the third attempt, a connection will be established at step 186. The algorithm further provides for receiving information from the patient facility at step 182. As indicated previously, the database at the patient facility will transmit an order over the link 172 from the transmit order step 170. This communication is introduced into the information received section 186 on the supplier side.

A determination is also made as to whether or not this is the third attempt to receive information at step 188. At this point, the algorithm will provide for selection of a method to handle the information at step 190. Thereafter that information is received at step 192 and the information is inserted into an order bank 194 at the data bank of the supplier. Thereafter, that data bank will cause the sending of a reply along with a tracking number at step 196 to a receive-reply determination 198 of the data bank at the patient facility. That receive-reply determination will determine whether or not a reply has been received from step 196 at the supplier facility and if so, will introduce the information into a database 200. If a reply has not been received, a determination will be made as to whether this is the third attempt at the step 174 and if so, then the algorithm will cause an automatic termination at step 176.

If the information has been received into the first database of the patient facility at step 200, a termination notice will be sent at step 202. That termination notice will be sent to a process to terminate step 204 of the supplier's database. Moreover, after the tracking number has been sent from step 196 to the database of the patient facility, that information is sent to step 206 which then connects to the process to terminate the linkage.

The algorithm continues with the process of waiting for a patient as shown at step 208 in FIG. 5B. At this point, several determinations are made involving the administration of the radioactive pharmaceutical to the patient. At the outset, there is a determination as to whether or not the proper dose is available at step 210 for administration to the patient. If the dose has been available, then the patient can be injected with the date and time and other related data taken at step 212 and recorded. If there has not been a receipt of the dose of the proper radioactive pharmaceutical, then a determination will be made as to whether or not the pharmacy has been contacted at step 214. If the pharmacy has not been contacted then the algorithm will loop, again, as to whether or not there is an available dose for injection. If the pharmacy has been contacted, then a contact will be made with the pharmacy for the status information at step 216.

After the patient has been injected with the radioactive pharmaceutical, or other means of application have been used for applying the radioactive pharmaceutical to the patient, the algorithm then proceeds with quality control operations at step 220 and, in this case, provides for disposal of the syringe. Thereafter, a test is performed with the patient to insure that the patient has the proper injection and the patient can then be removed at step 222. Further, an area wipe for radioactive contamination must then be conducted at step 224 and the data for that area wipe test must be at least recorded for the printing of reports at step 226.

It can be observed at this point that the algorithm is effective in actually recording all of the necessary information for record keeping, as for example, information regarding the injection of the patient, information regarding the monitoring of the area and the patient and the information regarding disposal of any implements used in connection therewith.

Turning now to the supplier side of the algorithm, if the supplier has received the termination signal at step 204, the supplier will then print the necessary documents to fill the order from the patient facility at step 228. At this point, it is to be noted that the communication is temporarily terminated between the two databases until such time as the order has been filled and delivered.

A determination is then made as to whether or not the prescribed radioactive pharmaceutical is in inventory at step 230. This implies not only does the pharmaceutical have the correct composition, but whether or not it also has the proper amount of radioactivity. If there is a determination at step 230 that the prescription is in the inventory, the order will be filled at step 234. If there is a determination that it is not in the inventory then the elute process will be started at step 236. In the elute process, the proper pharmaceutical must be prepared and thereafter irradiated so that it contains the proper amount of radiation particularly considering the amount of potential decay before delivery to the patient or user, e.g. patient facility.

The order is then sent at step 240 to the patient facility over a delivery link identified as 242 and a determination will again be made at step 210 on the database of the patient facility as to whether or not the dose is available for injection. If there is a dose available, the process will continue as described and if there is not the dose available, then the process will also follow the algorithm as previously described.

If the order has been completed and sent to the patient facility, the second database at the supplier side will provide notification that the order is completed at step 244. Again, an area monitor and wipe must be conducted at step 246. Disposal of a syringe may occur at step 248 and all of the data for that information must be recorded at step 250.

It can also be observed in connection with the present invention that the patient facility could literally send the syringe back to the pharmacy or other supplier for disposal. At that point, the pharmacy would then have to make arrangements for the proper disposal of the syringe, which could well be storage until radiation has completely decayed. Any conventional means, usually accepted by governmental agencies, must be used for disposal of any radioactive contamination.

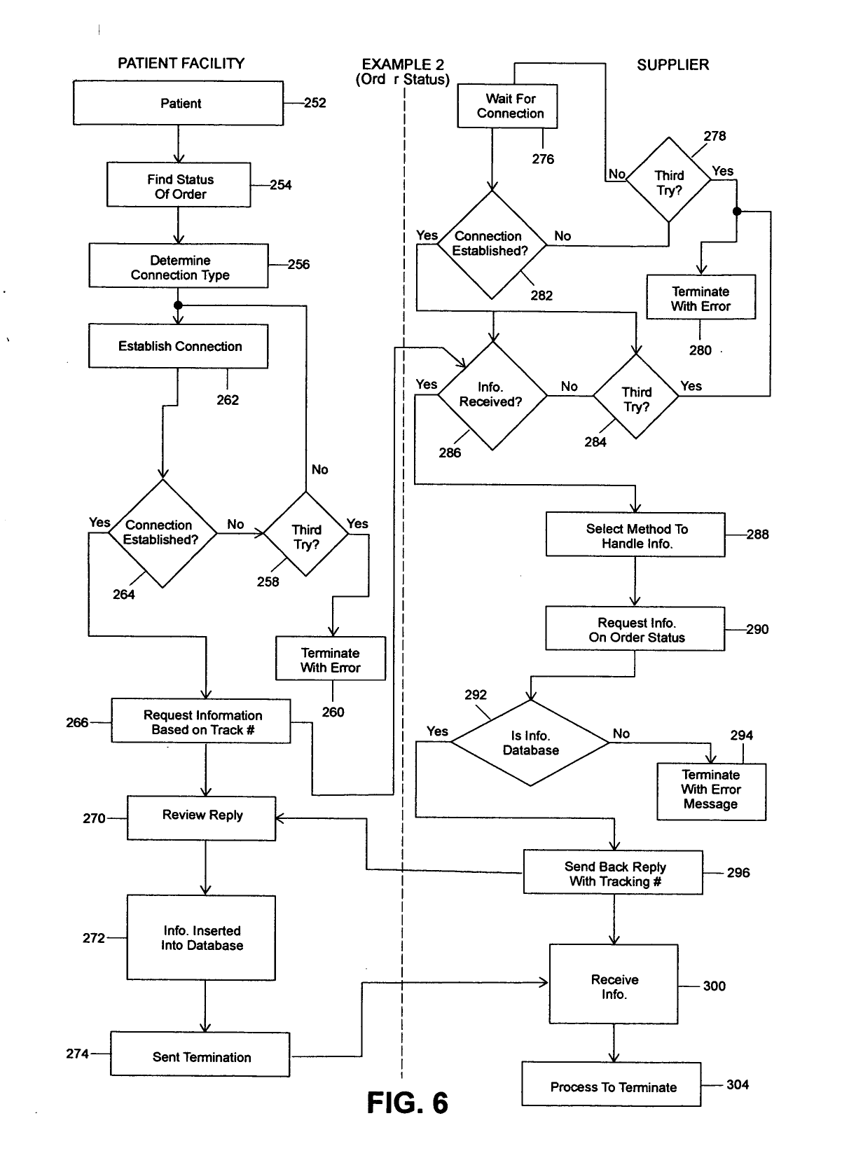

FIG. 6 illustrates an example, identified as Example 2, where inquiry is made of a status or an order at a database of a patient facility to a database of a supplier, such as a radioactive pharmaceutical supplier. In this case, the algorithm starts with information on the patient at step 252 along with an instruction thereafter to find the status of the order at step 254, assuming the order had been previously made. The algorithm proceeds to determine the type of connection which would be used depending on the mode of inquiry, at step 256.

At this point, the algorithm must then again determine whether there have been multiple attempts at this connection, as for example, the third connection at step 258. If this has been the third attempt, the algorithm will terminate with an error message at step 260. In addition, there will be an establishment of the connection at step 262 if this has not been the third attempt and there will then be a decision at step 264 as to whether the connection is established. If the connection is not established then again a decision is made at step 258 as to whether this is the third attempt. If not, the algorithm will proceed to step 266 providing for the requested information based on a tracking number. This request for information is sent to the supplier on the supplier side of the algorithm over the path 268, as shown in FIG. 6 of the drawings.

The user of the database at the patient facility will be desirous of knowing whether or not a reply is received from the supplier side of the algorithm at step 270. That reply, when received, is inserted into the database at step 272 and thereafter the process is terminated at step 274. In this case, the reply received at step 270 is the status of the order inquired by the patient facility. Further communication could continue with the same algorithm precisely in the form as described in this example.

Turning now to the supplier side of the algorithm, the supplier side waits for a connection as for example, a connection from the patient facility at step 276. Again, there is a decision at step 278 as to whether this is the third attempt. If it is the third attempt, then the algorithm terminates with an error message at step 280. If it is not the third attempt, a connection is established at step 282. Thereafter, another decision is made as to whether the connection is established and whether this is the third attempt at step 284. If it is the third attempt then the algorithm will cycle back to the terminate with error signal step 280.

The algorithm on the supplier side will determine if information is received at step 286 if this is not the third attempt. If the information has been received, the algorithm will cause the selection of the method used to handle information at step 288. Although the selection of the method to handle information has not been detailed at step 288, that selection is similar to, for example, steps 96, 98, 100 and 102 in the algorithm on FIG. 4B. In this case, the selection of the method would involve a decision step as well as three possible choices including the request for more information, termination or information to be returned to the database.

Termination can always be obtained and a request for more information can be made in the same manner as described in connection with this Example 2. In this case, the supplier merely adopts the position of the patient facility in the algorithm. With regard to the information returned to the database, there is a request for the information on the order status at step 290. A determination is made at step 292 as to whether that information is in the database. If it is not in the database, the algorithm will terminate with an error message at step 294. If that information is in the database, the algorithm will cause the sending of a reply with the tracking number to the patient facility at step 296. It is to be noted that that reply is received at a patient facility at step 270 over a data path 298. It is also to be noted that the request for information, based on a track number at step 266 in the patient facility, is introduced into the supplier facility at the decision point 286.

If and when the information has been sent and a reply has been received to the requesting patient facility, that information is inserted into the database 272 in FIG. 6, as aforesaid and a termination process will occur at the patient facility. That termination information is then received at step 300 on the supplier side of the algorithm. When the supplier side knows that the inquiry process has terminated at the patient facility, it will cause the process to terminate at the supplier facility at step 304.

It should be understood in connection with this example 2 of FIG. 6, that the direction of the communication paths could be reversed such that the supplier side inquires of the patient facility. In this case, there could also be a two-way communication in which the patient facility inquires of the supplier and thereafter the supplier inquires of the patient facility and this may continue. Again, the use of patient facility and the supplier has only been exemplary and any type of communication could take place. For that matter, a communication could exist in the manner as shown in connection with FIGS. 1 and 2 of the drawings, as well.

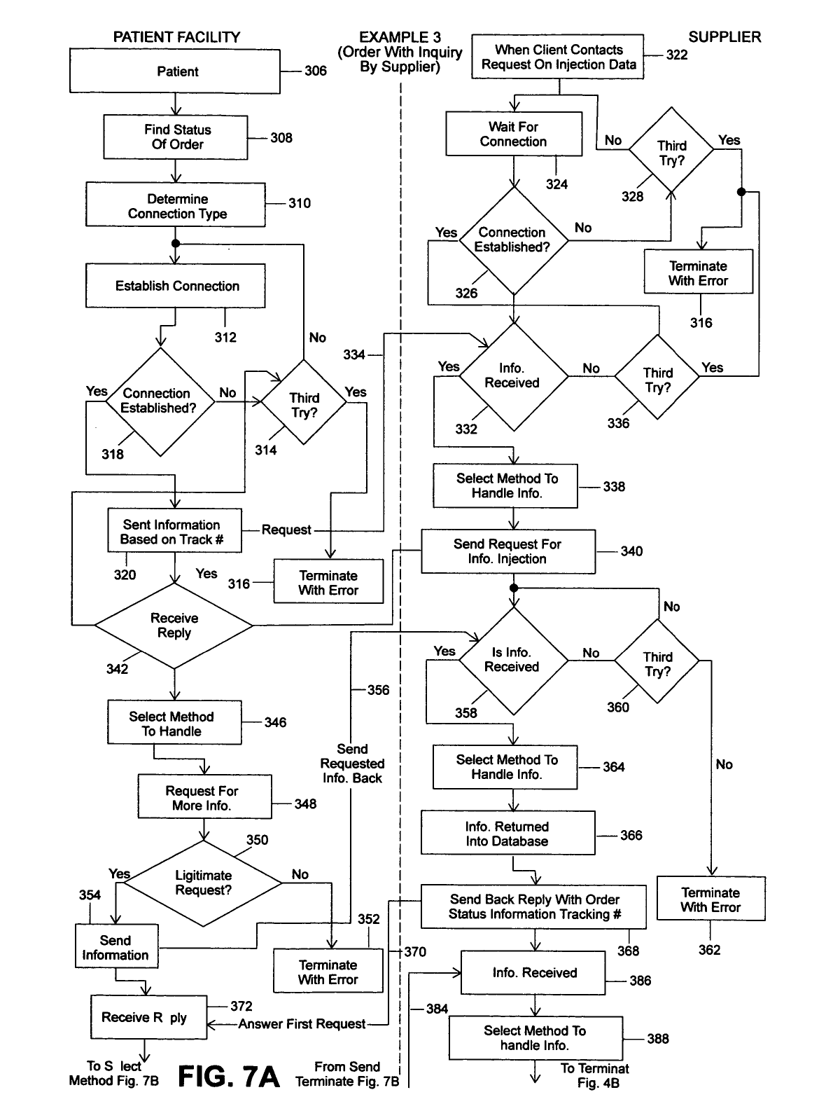

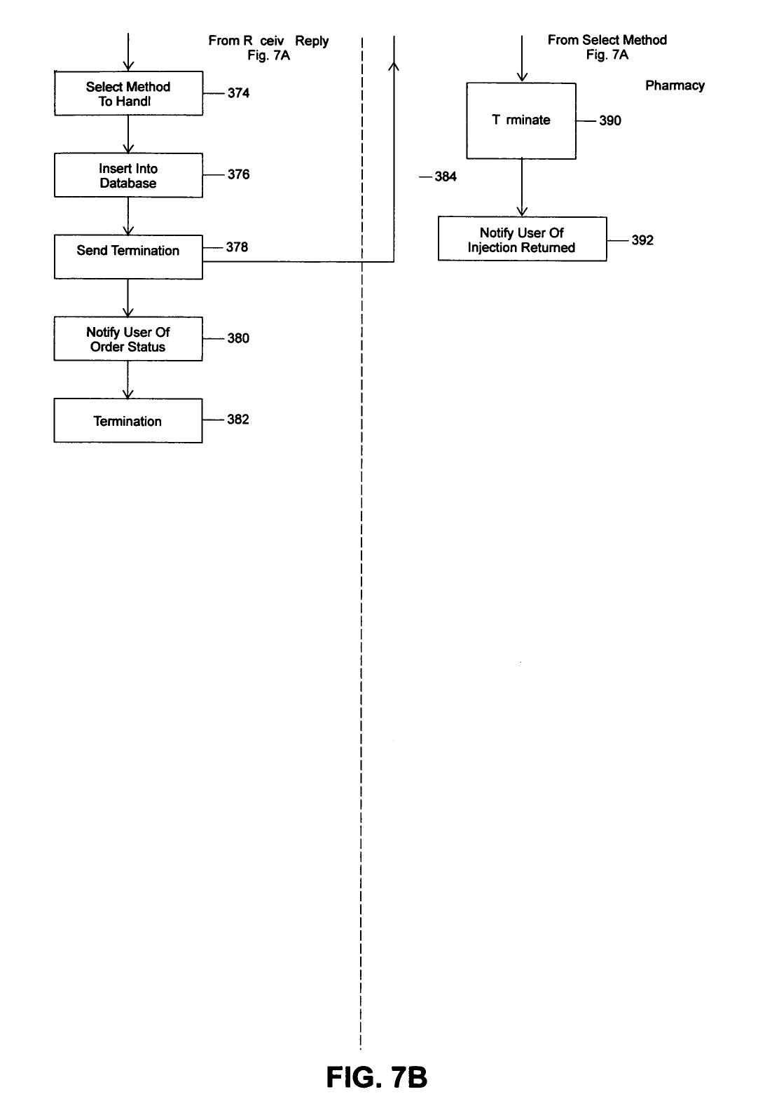

FIGS. 7A and 7B set forth a third example dealing with an order by a patient facility along with an inquiry by a supplier before the filling of that order. In this case, for example, a patient facility may order a radioactive pharmaceutical and the supplier may need further information before even filling that order. Thereafter, the supplier may inquire of the patient facility before taking any action and when the patient facility supplies the information required by the supplier, then the supplier can fill that order and send same to the patient facility.

The algorithm starts with information on a patient at step 306. At this point, someone at the patient facility may be desirous of the status of an order at step 306 and which order was made at a previous time. The algorithm will then proceed to determine the type of connection at step 310. Thereafter, a connection can either be established at step 312, but if this is the third attempt which is determined at step 314, the algorithm will cause a termination with an error signal at step 316. If it is not the third attempt, a connection will be established at step 318. When the connection is established, the request for information or otherwise an order will be sent to the supplier facility based on a tracking number at step 320.

Turning now to the supplier side, the algorithm provides a step 322 which is a wait for information upon contact by a third party as, for example, the patient facility. Thereafter, when a contact is made, as for example, from the patient facility, the algorithm will cause a waiting for a connection at step 324. When a connection is established at step 326, a determination will be made as to whether this is the third attempt at step 328. If this is the third attempt, then there will be a termination with error signal at step 330. If it is not the third attempt, then the information will be received from the patient facility at step 332. In this case, information is received from step 320 at the patient facility on the patient side of the algorithm over a data path 334. A determination is made at step 332 as to whether this information has been received. If not, an determination is made at step 336 as to whether this is the third attempt. Clearly if it is the third attempt there will be a termination with error signal at step 330 and if not, the algorithm will proceed to receive the information and continue with the algorithm in order to fill the order.

On the supplier side, the algorithm must also select a method to handle the information at step 338. As indicated previously, this selection of the method to handle the information is that shown by steps 96, 98, 100 and 102 in the example set forth on FIG. 4B.

In the embodiment of Example 3, there may be a situation where more information is needed from the supplier before even attempting to fill the patient facility order or before providing a response to an inquiry made by the patient facility. In this case, the database of the supplier side will cause the sending of a request for more information such as injection information, for example, at step 340 and this will be returned to a receive-reply step 342 on the patient facility side. Moreover, this request will be made over a data path 344. On the patient facility side of the algorithm there will then be a method selected to handle the request from the supplier side at step 346. The patient facility must also determine who will receive the request for more information at step 348 and will be then required to make a determination as to whether this is a legitimate request at step 350. If it is determined not to be a legitimate request, the patient facility will automatically cause a termination with an error signal at step 352. If it is a legitimate request, the patient facility will send the information requested by the supplier at step 354. This information in response to the request by the supplier is sent back to the supplier database on a data path 356.

This reply in response to the inquiry at the supplier facility is received at 358 in the supplier facility with a determination as to whether the information is received. If the information has not been received then a determination is made as to whether this is the third attempt at step 360. If it is the third attempt the algorithm will terminate with an error signal at step 362. If it is not the third attempt and the information has not been received the algorithm on the supplier side will proceed to step 364 which is again, a selection method to handle the information. That same selection procedure in FIG. 4B involving steps 98, 100 and 102 is employed for this purpose.

The information which is received from the patient facility in response to the inquiry by the supplier is then returned to a database at step 366. At that point, the supplier can send back a reply at step 368 over a data path 370. That answer is actually the answer to the first request made by the patient facility and is received at a receive-reply step 372 on the patient facility side of the algorithm in FIG. 7A. When the reply has been received from the supplier over the path 370 and at the step 372 on the patient facility side, then a method to handle that information is selected at step 374. Moreover, that information which constituted an answer to the request by the patient facility is inserted into the database at step 376. At that point, the algorithm on the patient facility side can then terminate the inquiry and/or order at step 378. Thereafter, the patient facility can notify the user at that facility of the order of the status at step 380 and terminate at step 382.

When the patient facility side of the algorithm terminates, a termination signal over a path 384 will be sent from the termination notification step 378 to an information received step at 386. At that point, the algorithm on the supplier side will select a method to handle information at step 388. Thereafter, the algorithm will cause a termination at step 390 and notify the user of the fact that the requested pharmaceutical has been returned at step 392.

It should be understood that the three examples constituting FIGS. 5A, 5B, 6, 7A and 7B are only representative of the types of communications which can be connected with the integrated algorithm of the invention. This algorithm obviously allows for numerous other types of communication. For example, complaints and for that matter even conferencing can be performed using the algorithm capable of allowing a two-way integrated communication. In effect, the algorithm allows for communication between databases much in the same manner as in communications between individuals.

Thus, there has been illustrated and describe a unique and novel algorithm which permits generation of a method for the integration, distribution and communication in the providing, handling and distributing of radioactive pharmaceuticals and generating reports therefor. The algorithm of the invention and the process generated thereby fulfills all of the objects and advantages which have been sought. It should be understood that many changes, modifications, variations and other uses and applications which will become apparent to those skilled in the art after considering the specification and the accompanying drawings. Therefore, any and all such changes, modifications, variations and other uses and applications which do not depart from the spirit and scope of the invention are deemed to be covered by the invention.

Having thus described the invention, what we desire to claim and secure by Letters Patent is:

1 Algorithm for establishing an electronic bi-directional communication between at least a first database and a second database for producing delivery and monitoring of an isotope irradiated nuclear medicine, said algorithm comprising:
a) sending information regarding the isotope irradiated nuclear medicine from a first database to a second database and which information may be data pertaining to the production or delivery of the nuclear medicine or the use thereof or in the handling and disposition of any implement used in connection therewith which might have been irradiated as a result thereof; and
b) terminating the sending of information by the first database.

2 The algorithm for establishing electronic bi-directional communication of claim 1, where said algorithm comprises:
determining whether the second database is authorized to access a data communication before sending such communication.

3 The algorithm for establishing electronic bi-directional communication of claim 2 further characterized in that said algorithm further comprises:
terminating and link between the first and second databases if the second database is not authorized to access such data communication.

4 The algorithm for establishing electronic bi-directional communication of claim 1 further characterized in that said second database is contained in a hand held portable electronic device.

5 The algorithm for establishing electronic bi-directional communication of claim 1 further characterized in that said algorithm further comprises:
causing said second database to send back a message to said first database that the data from said first database had been received after receipt thereof.

6 The algorithm for establishing electronic bi-directional communication of claim 5 further characterized in that said algorithm further comprises:
determining the correct communication link to be established between the first and second databases.

7 The algorithm for establishing electronic bi-directional communication of claim 5 further characterized in that the algorithm further comprises:
a) sending an inquiry for information from said second database to said first database; and
b) thereafter sending the information sought by said second database back to the second database.

8 The algorithm for establishing electronic bi-directional communication of claim 5 further characterized in that the algorithm further comprises:
a) sending an inquiry for information from said second database to said first database; and
b) holding a response to that inquiry in abeyance and making an inquiry by said first database to the second database and only sending that response if the first database receives a response first to its inquiry.

9 Algorithm for establishing bi-directional communication regarding an isotope irradiated medicine between at least a first database and a second database with an operator at least at one of said databases for dealing with an isotope irradiated nuclear medicine related item, said algorithm comprising:
a) a step for establishing a link between the two databases;
b) a step for transmitting information about the isotope irradiated nuclear medicine related item from a first database to a second database enabling a response from said second database back to said first database regarding the production, delivery or monitoring of the nuclear medicine or any implement used therewith which could have been irradiated;
c) electronically determining the mode of information handling at the second database;
d) a step for transmitting information from said first database to said second database regarding the nuclear medicine or any implement used therewith; and
e) a step for sending a termination message from at least the database where transmission is occurring to the other database.

10 The algorithm for establishing bi-directional communication of claim 9 further characterized in that said algorithm further comprises:
sending an information transmission termination signal from each of the databases to the other upon completion of the transmission.

11 The algorithm for establishing bi-directional communication of claim 9 further characterized in that said algorithm comprises:
a step to determine if the information being transmitted to the second database is already in the second database and disregarding that information if already present in the second database.

12 The algorithm for establishing bi-directional communication of claim 9 further characterized in that the algorithm further comprises:
a step to analyze if a request for information from one of the databases is an authorized request and to disregard that request if not authorized.

13 A process generating computer based algorithm for integrating the acquisition and transmission of data with respect to radioactive pharmaceuticals between a first database at a first location containing data relating to such radioactive pharmaceuticals and a second database at a second location containing data relating to such radioactive pharmaceuticals and which transmission of data also relates to use of such radioactive pharmaceuticals, said process generating algorithm comprising:
a) allowing for transmission of data regarding radioactive pharmaceuticals from the first database to the second database for a particular radioactive pharmaceutical;
b) electronically parsing that data at the second database into an appropriate database section therefor and assigning a code for that data; and
c) electronically sending confirmation back from the second database to the first database about receipt of the transmitted data such that a user of the first database is aware that such data was received by the second database and further allowing the second database to be aware of information at the first database sharing awareness of the received data to the first database.

14 The process generating algorithm of claim 13 further characterized in that the process generating algorithm also comprises:
a step for maintaining a quality control with regard to the radioactive pharmaceuticals.

15 The process generating algorithm of claim 13 further characterized in that the algorithm provides for ordering of a radioactive pharmaceutical by the first database and the transmission to the second database the data specifying a radioactive pharmaceutical to be ordered by the first database and the algorithm further comprises:
a) preparing the radioactive pharmaceutical requested by that order and arranging for delivery of same to a user at the first database who ordered the radioactive pharmaceutical; and
b) electronically informing the user at the first database of the fact that the ordered radioactive pharmaceutical is being delivered by a party at the second database.

16 The process generating algorithm of claim 15 further characterized in that said algorithm further comprises:
a) causing a monitoring of the radioactive pharmaceutical being delivered by the person at the second database to the user at the first database;
b) causing a monitoring of all implements used in connection with the radioactive pharmaceutical at the site of production or delivery associated with the second database; and
c) recording of data associated with said monitoring activities of steps (a) and (b) within claim 16 and separating the data into appropriate fields allowing for generation of reports therefrom.

17 The process generating algorithm of claim 15 further characterized in that the step of preparing the radioactive pharmaceutical further comprises:
a) withdrawing the pharmaceutical from an inventory thereof; or
b) electing the pharmacies to produce the same; and
c) maintaining data necessary to keep a record thereof.

18 The process generating algorithm of claim 17 further characterized in that the algorithm further comprises determining if the request by the person associated with the second database is authorized before producing that requested radioactive pharmaceutical and disregarding the request if not authorized.

19 A computer based process generating algorithm for integrating the acquisition and transmission of data between a supplier and a user in the generation and administration of radioactive pharmaceuticals, said process comprising:
a) allowing for transmission of an order to a pharmacy from a user for a particular radioactive pharmaceutical;
b) preparing that radioactive pharmaceutical and maintaining data therefor in an appropriate database;
c) assigning a confirmation for the preparation of that radioactive pharmaceutical;
d) selecting a delivery mode based on time of administration and decay time of the radioactive pharmaceutical;
e) delivering the radioactive pharmaceutical to the user by the selected delivery mode; and
f) electronically maintaining data of the delivery in said appropriate database.

20 The process generating algorithm of claim 19 further characterized in that said process also comprises:
sending information relating to the order and the delivery to a quality control person and integrating into a database of the quality control person.

21 The process generating algorithm of claim 20 further characterized in that said process also comprises:
sending information relating to the order and the delivery generally simultaneously with performance of steps (a) through (d) to a quality control person and integrating that information into a data base of the quality control person.

22 The process generating algorithm of claim 1 further characterized in that the process generating algorithm also comprises steps to be undertaken by persons in the:
a) maintaining a quality control in the preparing of the radioactive pharmaceuticals; and
b) maintaining a quality control in the delivery of the radioactive pharmaceutical.

23 The process generating algorithm of claim 22 further characterized in that the process generating algorithm also comprises the step of:
introducing information about the quality controls into an appropriate database to enable generation of reports based on such information.

24 The process generating algorithm of claim 22 further characterized in that the process generating algorithm also comprises the steps of:
generating an informational display regarding procedures for disposal of such radioactive pharmaceuticals and generation of reports with regard to such disposal.

25 The process generating algorithm of claim 24 further characterized in that the process generating algorithm also comprises the step of:
sending information relating to the order and the delivery to a health physicist and integrating such information into a database of the health physicist.

26 The process generating algorithm of claim 24 further characterized in that the process generating algorithm also comprises the steps of
a) maintaining a quality control in the preparing of the radioactive pharmaceuticals; and
b) maintaining a quality control in the delivery of the radioactive pharmaceutical.

27 A computer based algorithm for generating a process for integrating the acquisition and transmission of data used in the generation of a radioactive pharmaceutical by a supplier for a user of such radioactive pharmaceutical, said process generating algorithm comprising:
a) allowing for transmission of an order for a radioactive pharmaceutical to a supplier from a user for a particular radioactive pharmaceutical;
b) electronically parsing that order by the supplier into an appropriate database therefor and assigning a confirmation for that order;
c) electronically sending that confirmation back to the user;
d) electronically parsing information by the user into an appropriate database such that the user is aware that such order was received by the supplier and the supplier is aware of the users knowledge of receipt of the order;
e) preparing the radioactive pharmaceutical requested by that order and arranging for delivery of same to the user who ordered the pharmaceutical; and
f) electronically informing the user of the fact that the ordered radioactive pharmaceutical is being delivered.

28 The process generating algorithm of claim 27 further characterized in that the process generating algorithm also comprises:
sending information relating to the order and the delivery to a health physicist and integrating that information into a database of the health physicist.

29 The process generating algorithm of claim 27 further characterized in that the process generating algorithm also comprises:
a) maintaining a quality control in the preparing of the radioactive pharmaceuticals; and
b) maintaining a quality control in the delivery of the radioactive pharmaceutical.

30 The process generating algorithm of claim 29 further characterized in that the process generating algorithm also comprises a step for:
introducing information about the quality controls into an appropriate database to enable generation of reports based on such information.

31 The process generating algorithm of claim 27 further characterized in that the process generating algorithm also comprises a step for:
generating an informational display regarding procedures for disposal fo such radioactive pharmaceuticals and generation of reports with regard to such disposal.

32 The algorithm generating computer based process of claim 27 further characterized in that said process generating algorithm comprises a step for:
electronically sending information relative to the order to a health physicist and integrating such information into the database of the health physicist.

33 The algorithm generating computer based process of claim 27 further characterized in that said process generating algorithm comprises generally simultaneously with performing steps (a) through (f) also electronically sending information relating to the order to a health physicist and integrating such information into the database of the health physicist.

34 A computer based algorithm for generating a process for integrating the acquisition and transmission of data used in the generation of a radioactive pharmaceutical between a supplier and a user of such radioactive pharmaceutical, said process generating algorithm comprising:
a) allowing for transmission of an order for a radioactive pharmaceutical to a supplier from a user for a particular radioactive pharmaceutical;
b) electronically parsing that order by the supplier into an appropriate database therefor and assigning a confirmation for that order;
c) electronically sending that confirmation back to the end user;
d) parsing information by the user into an appropriate database such that the user is aware that such order was received by the supplier and the supplier is aware of the users knowledge of receipt of the order; and
e) also electronically sending information relating to the order to a quality control person and integrating into the database of the quality control person.

35 The algorithm generating computer based process of claim 34 further characterized in that said algorithm generating process comprises generally simultaneously with performing steps (a) through (f) also electronically sending information relating to the order to a health physicist and integrating such information into the database of the health physicist.
